(19) United States
(12) Patent Application Publication
(10) Pub. No.: US 2018/0140736 A1
(43) Pub. Date: May 24, 2018

(54) ORNAMENTAL AIR FRAGRANCE DISPENSER

(71) Applicant: Jack Webb, Frankfort, KY (US)

(72) Inventor: Jack Webb, Frankfort, KY (US)

(21) Appl. No.: 15/788,182

(22) Filed: Oct. 19, 2017

Related U.S. Application Data
(60) Provisional application No. 62/411,641, filed on Oct. 23, 2016.

(51) Int. Cl. A61L 9/12; A61L 9/04

(52) U.S. Cl. CPC A61L 9/12; A61L 9/042; A61L 9/044

(57) ABSTRACT

An ornamental fragrance dispenser for spreading fragrant aroma in selected areas. The air freshener filter insert is a strip of permeable solid strip of material impregnated with a time release substance of selected fragrance. An air freshener strip of selected dimensions is disposed in a cooperatively engaging slot formed in the dispensing device mountable to an air vent of a vehicle of air displacement device such as a fan or suspended in a selected area by hanging means. The dispenser is a perforated housing or shell including a void shaped to receive and hold an absorbent strip containing time release liquid with natural oils which evaporate to spread a pleasant aroma in the surrounding area. The shell includes hanging means such as a hook, eyelet and cord, sticky tape and clip for hanging the shell from a car mirror, air vent, ceiling fan, kitchen appliance such as refrigerator, dishwasher, gym locker, and so on.

This application claims priority from U.S. Provisional Patent Application 62/411,641 filed on Oct. 23, 2016, which is incorporated by reference herein in it's entirety.

### TECHNICAL FIELD

The present invention relates to the field of air fresheners and air fragrance dispensers.

### BACKGROUND OF THE INVENTION

Air fresheners have been known in the art and are limited in their use. In many types of living space, but particularly in the passenger compartment of cars and trucks, there are often unpleasant odors. Such odors as that of stale tobacco smoke or odors of some material being carried in the passenger compartment can offend to many people. Often that offensive odor is treated by use of hanging devices having other perfumes or deodorant material absorbed into the device to be hung in the car. These devices are most often hung from the rear-view mirror or the sun shades in the vehicle. Typical air fragrance dispensers which are clipped on a car air vent are intended for car use only. Other dispensers have an attached string for hanging from rear view mirrors or other objects. Some are designed to hang from ceiling fans. Other examples are designed to sit on a shelf, table or other level surface.

Recent examples of air fragrance dispensers are powered by a regular wall outlet and include a heating element which helps to evaporate the fragrant volatiles. In some applications, these dispensers allow a user to refill the device with fragrant oils from a bottle.

This invention provides a simple and much more efficient way of distributing the sort of material that is absorbed into the former hanging devices. It also avoids any dangling object which might be distracting to the driver of an automobile. Further, it is inserted to be non-intrusive in outer space.

The need for an inexpensive and convenient to use air freshener to take advantage of forced air in an air grill has long been recognized. It has been proposed to provide an automobile air vent with wide spaced louvers for receipt thereon of a releasable fastener so that a container containing a deodorant may be mounted to the louvers.

U.S. Pat. No. 6,309,715 sets forth several patents which describe methods of incorporating a slow sustained release of a fragrant molecule as follows: U.S. Pat. Nos. 5,525,588; 5,525,555; 5,490,982; 5,372,806 describe dissolving or suspending fragrance compounds in emulsions. U.S. Pat. Nos. 5,500,223; 5,324,444; 5,185,155; 5,176,903; and 5,130,171 describe encapsulation of a flagrance. U.S. Pat. No. 5,234,689 describes dissolving a fragrance into a hydrophilic phase such as silicone; U.S. Pat. Nos. 5,387,622 and 5,387,411 incorporation of a fragrance into a cross-liked polymer. U.S. Pat. Nos. 5,071,704 and 5,008,115 incorporate a fragrance into a permanent laminate. U.S. Pat. No. 4,908,208 incorporate a fragrance that softens at body temperature. U.S. Pat. Nos. 4,524,018 and 4,500,725 describe incorporation of a fragrance into silanes with fragrant alcohol to form alkoxysilanes. U.S. Pat. No. 6,054,547 describes the incorporation of fragrant moieties via hydrosilation of an olefinic silane molecule. U.S. Pat. No. 5,034,222 describes a solid composite air freshening article in a granular foam phase dispersed throughout a gelled phase. U.S. Pat. No. 6,063,365 describes an emulsifier-free nonporous, continuous permeable polymeric film having an entrapped and dissolved fragrance capable of evolving from the film into the environment by means of molecular diffusion in a sustained and controlled release manner. The disclosure of the above U.S. patents are hereby incorporated by reference herein as if set forth in their entirety.

An air freshener strip filter insert generally consists of a substrate impregnated with a substance that is time released into the surrounding air and in which such substance, when in the air, can, upon appropriate choice, provide a pleasant aroma. Air fresheners are available that are attachable to the grill of an air outlet of an air ventilating system so as have the air freshening substance added directly to the air flowing into a vehicle cabin or they maybe passive and merely consist of a substrate or carrier containing an aroma producing substance and fixed or attached to some part of the vehicle within the occupant compartment.

### SUMMARY OF THE INVENTION

In accordance with the present invention, there is provided an air fragrance kit comprising, consisting of, or consisting essentially of an ornamental fragrance dispenser housing, a number of fragrance pads, a selected length of cord, a clip, and a plurality of tacky strips. The dispenser housing includes a generally hollow shell whose outer walls are perforated over most of the surface area and which has a slot formed therein which can receive and hold a fragrance pad. The dispenser housing includes a hook at the top central edge thereof and a flat surface on the bottom side thereof. The fragrance pads are capable of holding a fragrant liquid and emitting a fragrance for up to 75 days. The selected length of cord is for attaching the hook of the housing to a selected object. The clip includes a post with a knob on the first end and on the second end are two outwardly extending cooperatively engaging jaws integral with the post and naturally urged against one another. The clip is capable of being urged over an edge of a vent louver. The diameter of the knob is slightly larger than the diameter of an aperture formed in a face of the dispenser housing. Each one of the plurality of tacky strips is suitable for attaching the dispenser housing to a selected surface.

It is an object of this invention to provide a fragrance dispenser kit which can be used to improve the smell of living areas in homes, commercial areas, vehicle interiors, and anyplace where it is desired to improve the smell and perhaps to mask over other undesirable odors.

A principal object of the present invention is to provide a replaceable air freshening device for an occupant compartment air circulating system of a motor vehicle.

A further object of the present invention is to provide an air freshening device that is readily customized to fit various different air circulating systems and particularly ones having an air flow through filter therein

Another object of the present invention is to provide an air freshener device having high surface area capable of being disposed within the pleats of a conventional particulate filter to maximize the surface area of the fragrance filter and utilize the structural support of the conventional pleated particulate filter.

It is an object of this invention to provide a fragrance dispenser kit which allows a user to insert a fragrance refill pad into a slot in an ornamental housing and then apply a cord, clip or tacky strip to the housing in order to hang, clip or adhere the housing to any desired object.

It is a further object of the present invention to provide a disposable flagrance cabin air filter means wherein a particular scent or aroma can be distributed throughout the (ventilation and) ventilated cabin area and provide a means for aroma therapy with selected scents including fruit and berry scents, such as evergreen scents, citrus scents, vanilla, and spice scents.

It is a further object of the present invention to provide a disposable flagrance cabin air filter means wherein a disinfectant, deodorant, biocide, deodorant, bactericide, fungicide, mold, and/or mildew retardant or the like can be distributed throughout the ventilation and cabin area in order to purge the ventilation system and be removed or retained in an amount to retard contamination by same.

It is an object of this invention to provide a fragrance dispenser kit including an ornamental housing or shell with a slot or void which receives and holds a pad containing fragrant oils.

It is an object of this invention to provide a fragrance dispenser kit including an ornamental housing or shell with a flat surface situated at a bottom edge which allows the dispenser to be self supporting while allowing the ornamental face of the dispenser to be displayed in an upright orientation.

It is an object of this invention to provide a fragrance dispenser kit which includes a combination hook and eyelet for cord or string attachment for the purpose of hanging the dispenser, a tacky strip for the purpose of sticking the dispenser to a selected surface such as an appliance door, and a clip which can be attached to one side of the dispenser which then allows the dispenser to be clipped to the edge of a vent louver or the edge some other object.

It is an object of this invention to provide a fragrance dispenser kit which includes refill strips which contain the fragrance oil and which can be used to replace spent refill strips in the dispenser.

It is an object of this invention to provide a fragrance dispenser kit which includes refill strips which can have selected fragrance oils applied by a user and then be inserted into the dispenser.

It is an object of the present invention to provide an air fresher dispenser comprising a housing having an ornamental shape including an airflow passage providing an air flow through substrate having an air freshening substance releasably retained therein and locating the same across said airflow passage at a location between an inlet to said airflow passage and an outlet therefrom.

It is an object of the present invention comprising a base strip of porous material having an air freshening substance releasably retained therein, and means retaining said particulate material on said base strip.

It is an object of the present invention wherein the dispenser includes an elongate pocket having a top surface, a bottom surface connected with opposing sides walls and an end wall for receiving said strip, said dispenser including a plurality of perforations through said opposing sidewalls of said pocket providing cross-sectional air flow through said opposing sidewalls and around said strip inserted therein.

It is an object of the present invention wherein said elongate pockets extend in a direction transverse relative to the length of said base strip.

It is an object of the present invention wherein said insert comprises an acetate material.

It is an object of the present invention, wherein said strip is impregnated with a slow time release substance having a preselected fragrance.

Other objects, features, and advantages of the invention will be apparent with the following detailed description taken in conjunction with the accompanying drawings showing a preferred embodiment of the invention.

### BRIEF DESCRIPTION OF THE DRAWINGS

A better understanding of the present invention will be had upon reference to the following description in conjunction with the accompanying drawings in which like numerals refer to like parts throughout the views wherein:

FIG. 1 is a front view of a fragrance dispenser with a cord attached to the dispenser hook for hanging;

FIG. 2 is a top perspective view of a fragrance dispenser showing the slot for the fragrance pad;

FIG. 3 is a front view of a fragrance strip;

FIG. 4 is a front view of a fragrance strip with perforations;

FIG. 5 is a front view of a fragrance dispenser with a tacky strip applied to one side; and

FIG. 6 is a front view of a clip.

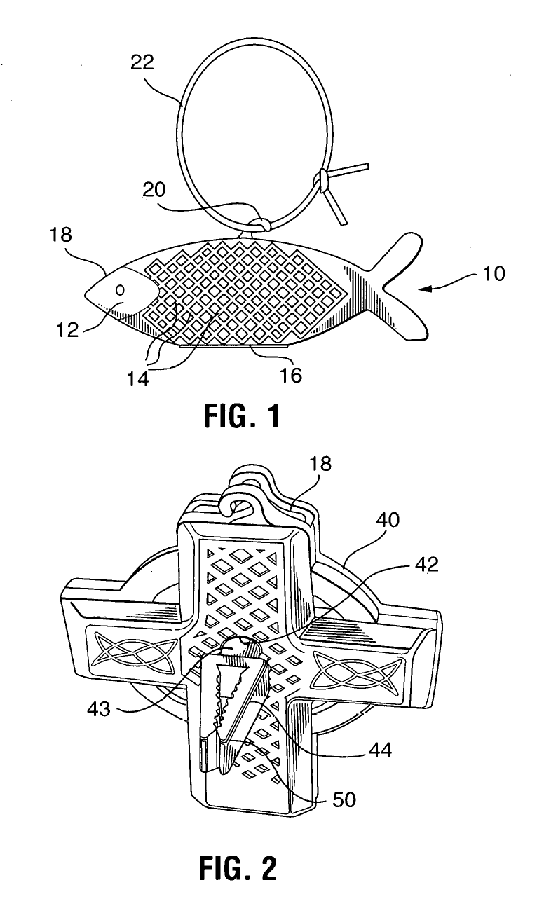

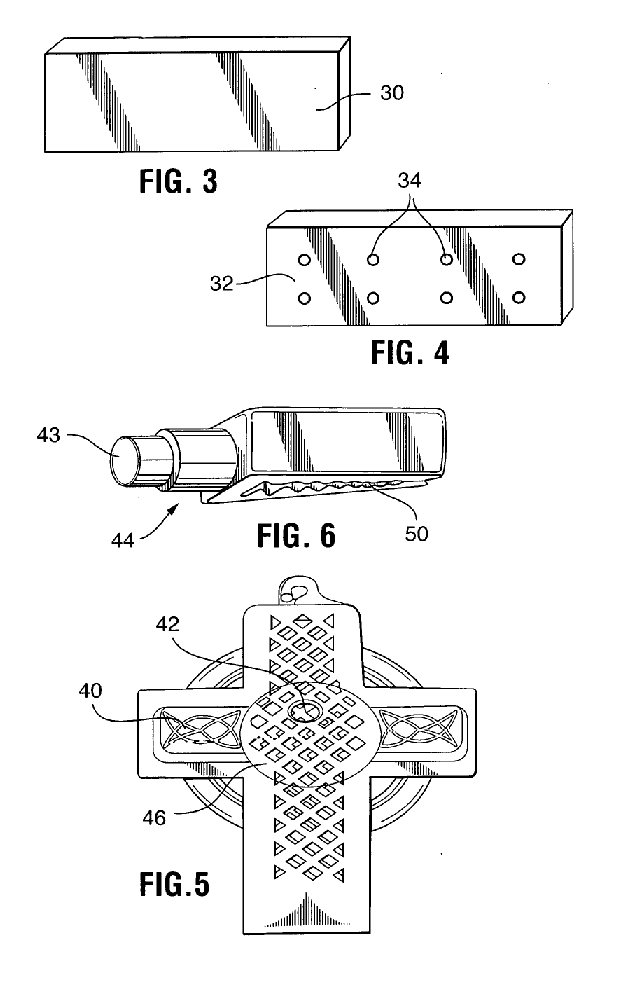

### DESCRIPTION OF THE PREFERRED EMBODIMENTS

The term air freshener as used throughout herein is intended to be generic to fragrance producing substances as well as disinfectants and the like that can be transferred to a flowing stream of air so to improve the quality of such air.

The air freshener and the present invention is a full air flow through device consisting generally of a substrate impregnated with a substance that is time released into the surrounding air and in which such substance when in the air can upon appropriate choice provide a pleasant aroma.

A particular scent or aroma can be distributed throughout the ventilation and cabin area and provide a means for aroma therapy with selected scents and fragrances. The fragrance oil can be any natural substance, synthetic material, (incorporating aldehydes, ketones, esters, and other chemical constituents), or combinations thereof which is known in the art and suitable for use in candles for imparting an odor, aroma, or fragrance. Suitable natural and synthetic fragrance/flavor substances include those compiled by the U.S. Food and Drug Administration in Title 21 of the Code of Federal Regulations, Sections 172.510 and 172.515 respectively. Suitable fragrances include spice oil, flower oil, and fruit oil. The fragrance oil may contain fragrance components, for example benzaldehydes, phenols, cinnamic aldehydes and esters, octadienes, dienes, cyclohexadienes, and terpenes.

The fragrances may comprise an essential oil in a carrier such as water and/or alcohol or other organic solvent or even a perfume. The fragrance may be that of a fruit and berry scents such as: citrus, almond, apple, cherry, grape, pear, pineapple, orange, strawberry, raspberry, and musk; flower scents such as lavender, rose, iris, carnation, gardenia, tea rose, violet, hyacinth, magnolia, mimosa, honeysuckle, jasmine, narcissus, orange blossom, orchids, sweet pea, tuberose, and lilac; forest and herbal smells such as evergreen cedar, pine, sassafras, and spruce; essential oils such as spice, peppermint, vanilla, spearmint; and various other fragrances such as leather, new car odor, acacia, cassie, cypre, cyclamen, fern, hawthorn and the like. The fragrance is not critical so long as it is compatible with the polymer impregnated with the time release carrier. The level of fragrance can vary up to about 100 percent by weight and more preferably from 0.001 to about 100 percent by weight. Other agents such as surfactants, emulsifiers, and polymers can be used to encapsulate the fragrance.

The carrier and means of controlling the release of the selected fragrance is dependent upon many variables including time and temperature with respect to the environment inside and outside of the vehicle. The fragrance and substrate composition comprising of 10% to 99.5% by weight of a suitable substrate and from 0.5% to 90% by weight of a fragrance oil.

It is also contemplated that the scented material may be composed of 100 percent of the fragrant raw material without a solvent or essential oil. Furthermore, the fragrance can be imparted to the air freshener filter absorbent material as an aerosol or gas vapor separate from, or in combination with a liquid substrate.

In one preferred embodiment, the fragrance can be absorbed by a polymeric material such as an ethyl vinyl acetate polymer simply by being disposed in close proximity thereto. Ethyl vinyl alcohol resin is another polymer having good scent absorbent capabilities.

One preferred group of copolymers of ethylene and vinyl acetate which absorb odors are produced by DuPont Industrial Polymers under the trade name of ELVAX resins which generally range in vinyl acetate content from 9 to 40% and have melt indexes from 0.3 to 500 dg/min. Moreover, the ELVAX resin from DuPont does not incorporate a plasticizer which could interfere with the scent absorbing capability of the polymer. The polymer can be formed in any desired shape prior to treatment or under low heat conditions (70.degree. C. or less). The ELVAX resin can be extruded or molded and blended with polyethylene, polypropylene, ABS resins, thermoplastic rubber nitril rubbers, natural rubber and other elastomers.

The amount of scent adsorbed into the vinyl acetate substrate and the control release mechanism is dependent upon the time and temperature relationship between the scent and substrate as well as the volatility of the scent, content or density of the polymer, level of polymerization, surface area (porosity), and pore size and structure of the polymer substrate which in the instant example is a vinyl acetate absorbent material. Thus, the impregnation occurs through passive adsorption and the time release mechanism occurs through passive diffusion.

As described in U.S. Pat. No. 2,169,055 by Overshiner et al., a cellulose compound such as cellulose acetate or cellulose nitrate can be imparted with a compatible scent imparting material which is released over an extended period of time. The cellulose compound can be produced in a solution with an organic solvent such as acetone and 1,4 diethylene oxide, and adding a scent imparting essential oil to the solution. Plasticizers such as diethyl phthalate and tri-acetic acid ester of glycerin may be used to impart flexibility to the material. The solution may be formed in to sheets, bars, films or the like by casting or molding. The solvents evaporate and a porous cellular structure is formed which releases the essential oil at a rate to provide an odor lasting for several months. Moreover, as shown in FIG. 6, the scent impregnated polymer is over wrapped and crimped at desired intervals to form an air tight seal utilizing a nonpermeable vacuum sealed material to prevent premature release of the scent in storage. Thus, the product can be air evacuated, or even vacuum sealed, in order to prevent release of the scent from the polymeric substrate prior to installation. The strength of the scent may also be determined by the quantity of the individual polymer units, (bars or particle containing pockets), which are cut from the roll and disposed in the filter system. Thus, the customer can decide upon the strength of the air freshener filter insert by using multiple packs cut to length. The utilization of sealed polymer substrates containing scents provides a method of prolonging and maintaining the shelf life and consistency of the time release product.

Micro encapsulation provides a method of controlling the release of scent in liquid form by enclosing the scent within hollow shells of differing size and wall thickness which can be dissolved or ruptured at different intervals to provide a generally steady supply of scent exposed to the environment. Moreover, semipermeable shells which allow escape through the shell wall without shell rupture exist to control the release of a scent.

Another method of controlling the release of the scent from a carrier is to use a homogeneous semipermeable material containing the active ingredient as a pure impregnate, solute or precipitate. The semipermeable material serves as the carrier from which it can only slowly escape by solution, diffusion, evaporation or combinations thereof. The characteristics of the carrier material depends on properties such as pore size, compatibility with the environment, liquid content, temperature of environment, wet-ability, and processing parameters.

One type of polymer liquid composite material prepared which can be utilized in the present invention is an organic or inorganic cellulose ester such as cellulose triacetate or cellulose nitrate as vehicles for the controlled release of active materials into the environment. As described in U.S. Pat. No. 3,985,298 by Nichols and incorporated by reference herein, the composite material can be formed to prepare transparent coherent materials formed as films, fibers or microspheres. The scent may be carried in a fluid which may comprise water, alcohol, ether, aliphatic and aromatic hydrocarbons, ketones, esters, and combinations thereof together with other chemical constituents. For instance, the chemical composition comprising the selected fragrance or scent or other chemical such as a disinfectant or mildew or fungicide may be incorporated as a component in an aerosol propellant, gas, or liquid containing a solvent carrier such as water and/or alcohol together with an essential oil having a selected fragrance or perfume for impregnation into the polymer-liquid composite. These polymer-liquid composite materials are prepared to incorporate interconnected internal pores from about 1 to 500 microns. These polymer-liquid composites often possess oleophilic hydrophobic surfaces and can contain release retarding gums and oils from which hydrophilic and oleophobic vehicles can be expelled by surface forces. Moreover, active ingredients can be incorporated in polymer-liquid composites as soluble particles or precipitates formed by solvent exchange or chemical reaction in situ to provide an internal reservoir which maintains a constant concentration of the active ingredient in solution inside the polymer-liquid composite. Thus, the polymer-liquid composite provides a means for zero-order release in which a nearly uniform level of active ingredient (scent) is maintained throughout the active life of the vehicle. The polymer-liquid composite material provides a means of retarding the evaporative release of volatile materials though diffusive effects and control of effective surface area, as well as through depression in the vapor pressure of the volatile substance. Furthermore, the polymer-liquid composite can be modified by skinning, by coating with a liquid to impede escape of the active ingredient (scent) or by the addition of a release-promoting agent to the environment.

In accordance with the present invention, there is provided a vehicle cabin air flow dispenser having a flow through strip having an air freshener filter insert strip or element contained therein. The strip is for use in association with a vehicle's conventional air filter of the vehicle's air conditioning and ventilating system. The air flow through strip comprises a base strip of porous material having a pocket. The pocket holds captive therein a selected quantity of scented particulate material. The scented particulate material comprising the fragrant element is composed of beads of a polymer such as an acetate material impregnated with a substance having a preselected scent. The porosity of the material is obviously co-related to the particle size of the particulate material so as to retain the particulate material on the base strip.

In the foregoing embodiment, the pockets are separately formed filled with a selected quantity of fragrance impregnated (beads) acetate beads or the equivalent. Moreover, flakes, strips of material, or solid bars may be used to form bars, strips, or wedges which may be disposed within the pockets.

The fragrant elements are molded or otherwise formed of a scented material and typically comprises a porous material such as an acetate polymer impregnated with a scent. The freshener element is enclosed in a porous means for holding such as a pocket or the like. It is also contemplated that the fragrant element may be formed of a soft pliable material such a highly plasticized polymer or material held in pockets or other means of retaining forming a frame. An important consideration is to provide as much surface area as possible to maximize the exposure of the air to the air freshener filter insert element.

In accordance with the present invention, there is provided a fragrance dispenser kit which includes, among other things, a fragrance dispenser 10 which comprises a housing or generally hollow shell whose outer walls are perforated over most of the surface area and which includes a slot or void which can receive and hold a fragrance pad 30 which is initially saturated with a fragrant liquid containing fragrant oils which will evaporate over time and provide a pleasant aroma in the living area surrounding the dispenser. The perforations 14 provide for extra evaporation of the fragrant liquid in the pad 30. The dispenser housing 12 includes a flat bottom surface 16 which will support the housing on a level surface such as a table top in such a way that the ornamental shape, such as a fish outline or a cross, are displayed in an upright configuration. The dispenser housing 12 includes a slot or void 18 into which is placed a fragrant pad 30.

Besides the fragrant dispenser 10, the kit includes a number of refill fragrance pads 30 and three choices of means of attaching the dispenser to a selected object. These attachment means include a cord 22 to be attached to a hook or eyelet 20 for hanging the dispenser 10 from some selected object, a number of tacky strips 46 for adhering the dispenser to the surface of a selected object such as the side or top of a kitchen appliance or any other selected object, and a clip 44 which can be attached to the dispenser 10 and then attached to the edge of one of the louvers of an air vent or the edge of some other object.

The included refill pads 10 may be provided with fragrance or may be neutral, allowing the user to treat the pads with a liquid fragrance of his own choosing. The fragrance pads are capable of holding and emitting a fragrant liquid for up to 75 days. One embodiment includes fragrance pads 32 with a plurality of apertures 34 to increase the surface area of the pad and therefore improve the evaporation rate of the liquid fragrance.

The tacky strip 46 in FIG. 5 is a transparent disc. Only one tacky strip 46 is necessary to adhere the dispenser to a surface, but several are included to provide spares. The strips are preferably a resilient material in the shape of a disc or a square with a tacky substance applied to each side thereof. Each tacky side is then covered with a piece of non-sticky film such as waxed paper or the like, to preserve the spare tacky strips for later use.

The clip 44, shown in FIG. 6 includes a post with a knob 43 on one end and on the other end are two outwardly extending cooperatively engaging jaws 50 which are integral with the post and which are naturally urged against one another and which can be urged over the edge of a vent louver or the edge of some other object. In this position, the two jaws firmly grasp the louver to hold the clip 44 and the attached dispenser 10 firmly to the louver. The diameter of the knob 43 is slightly greater than the diameter of an aperture 42 in one face of the dispenser 10 such that, a user can force the know 43 into the aperture 42 and the clip 44 will be held fast to the dispenser 10.

Therefore, any one of the three attachment means can be used by the user. It is preferred that the dispenser 10 is positioned in an area with air movement to help spread the pleasant aroma.

The foregoing detailed description is given primarily for clearness of understanding and no unnecessary limitations are to be understood therefrom, for modification will become obvious to those skilled in the art upon reading this disclosure and may be made without departing from the spirit of the invention and scope of the appended claims. Accordingly, this invention is not intended to be limited by the specific exemplification presented herein above. Rather, what is intended to be covered is within the spirit and scope of the appended claims.

I claim:

1. An air fragrance kit comprising:
an ornamental fragrance dispenser housing including a generally hollow shell whose outer walls are perforated over most of the surface area and which has a slot formed therein which can receive and hold a fragrance pad, said dispenser housing including a hook at a top central edge thereof and a flat surface on a bottom side thereof;
a number of fragrance pads capable of holding a fragrant liquid and emitting a fragrance for up to 75 days;
a selected length of cord for attaching said hook of said housing to a selected object;
a clip including a post with a knob on a first end and on a second end are two outwardly extending cooperatively engaging jaws integral with said post and naturally urged against one another and capable of being urged over an edge of a vent louver, a diameter of said knob being slightly larger than a diameter of an aperture formed in a face of said dispenser housing; and
a plurality of tacky strips suitable for attaching said dispenser housing to a selected surface.

2. The air fragrance kit defined in claim 1 wherein said number of fragrance pads includes a number of pads pre-treated with a fragrant liquid and a number of un-treated fragrance pads.

3. An air fresher dispenser comprising a housing having an ornamental shape including an airflow passage providing an air flow through substrate having an air freshening substance releasably retained therein and locating the same across said airflow passage at a location between an inlet to said airflow passage and an outlet therefrom.

4. The air freshener dispenser of claim 3, comprising a base strip of porous material having an air freshening substance releasably retained therein, and means retaining said particulate material on said base strip.

5. The air freshener dispenser of claim 3, said dispenser including an elongate pocket having a top surface, a bottom surface connected with opposing sides walls and an end wall for receiving said strip, said dispenser including a plurality of perforations through said opposing sidewalls of said pocket providing cross-sectional air flow through said opposing sidewalls and around said strip inserted therein.

6. The air freshener dispenser of claim 5, wherein said elongate pockets extend in a direction transverse relative to the length of said base strip.

7. The air freshener dispenser of claim 3, wherein said insert comprises an acetate material.

8. The air freshener dispenser of claim 3, wherein said strip is impregnated with a slow time release substance having a preselected fragrance.
